(19) United States
(12) Patent Application Publication
(10) Pub. No.: US 2018/0333560 A1
(43) Pub. Date: Nov. 22, 2018

(54) SYSTEM FOR CONTROLLED DEPLOYMENT OF MEDICAL DEVICES

(71) Applicant: Cook Medical Technologies LLC, Bloomington, IN (US)

(72) Inventor: Keith R. Milner, West Lafayette, IN (US)

(73) Assignee: Cook Medical Technologies LLC, Bloomington, IN (US)

(21) Appl. No.: 15/978,628

(22) Filed: May 14, 2018

Related U.S. Application Data
(60) Provisional application No. 62/508,154, filed on May 18, 2017.

(51) Int. Cl. A61M 25/01; A61M 25/09

(52) U.S. Cl. CPC A61M 25/0136; A61M 25/0113; A61M 25/09041

(57) ABSTRACT

A handle is provided for advancing a wire through a catheter, the handle including a handle body, a first wheel, a second wheel, and a coupling mechanism. The handle body has an outer surface and an inner surface. The inner surface defines a lumen extending along a longitudinal axis from a proximal end to a distal end. The first wheel is coupled to a first side of the handle body. A portion of the first wheel is movable toward the longitudinal axis into a first lumen side of the lumen to an engagement position to contact the wire. A second wheel is coupled to a second side of the handle body. A portion of the second wheel is adapted to extend into a second lumen side of the lumen which is different from the first lumen side.

### CROSS-REFERENCE

The present application is a continuation of and claims priority to U.S. Provisional Patent Application 62/508,154, “System for Controlled Deployment of Medical Devices” filed on May 18, 2017, which is incorporated by reference in its entirety.

### TECHNICAL FIELD

This disclosure relates to delivery devices for endoluminal medical devices and in particular, to control systems for endoluminal deployment of medical devices by delivery wires.

### BACKGROUND

The statements in this section merely provide background information related to the present disclosure and may not constitute prior art.

Medical devices, such as embolization coils or stents, are frequently delivered endoluminally to various areas of the body. Particularly in narrow passages, such as when delivering a neurovascular stent, microcatheters are used to reach delivery sites through narrow and tortuous vasculature. Delivery wires are used in conjunction with microcatheters to push the devices out of distal end of the microcatheters and deploy the devices. However, these delivery wires are thin and vulnerable to kinking if excessive force is used to advance the delivery wire. A kink in the delivery wire prevents deployment of the device. In some cases, a kink in the delivery wire may be corrected by partially retracting the delivery wire or microcatheter, but this increases the duration of the procedure. Therefore, a control system for precisely controlling the advancement and retraction of the delivery wire and catheter is desirable.

### SUMMARY

Further areas of applicability will become apparent from the description provided herein. It should be understood that the description and specific examples are intended for purposes of illustration only and are not intended to limit the scope of the present disclosure.

In one embodiment, a handle is provided for advancing a wire through a catheter, the handle including a handle body, a first wheel, a second wheel, and a coupling mechanism. The handle body has an outer surface and an inner surface. The inner surface defines a lumen extending along a longitudinal axis from a proximal end to a distal end of the handle body. The first wheel is coupled to a first side of the handle body. A portion of the first wheel is movable toward the longitudinal axis into a first lumen side of the lumen to an engagement position to contact the wire. A second wheel is coupled to a second side of the handle body. A portion of the second wheel is adapted to extend into a second lumen side of the lumen which is different from the first lumen side. The coupling mechanism is at the distal end of the handle body and is adapted to detachably secure the handle body to the catheter.

In another embodiment, a system for deploying a medical device through a catheter is provided including a catheter, a handle, a medical device, and a wire. The catheter has a lumen. The handle includes a handle body having an inner surface defining a lumen about a longitudinal axis extending from a proximal end to a distal end of the handle body. The handle also includes a first wheel, a second wheel, and a coupling mechanism. The first wheel is movable toward the longitudinal axis into a first lumen side of the lumen to an engagement position. The second wheel is coupled to a second side of the handle body. A portion of the second wheel is adapted to extend into a second lumen side of the lumen. The coupling mechanism is at the distal end of the handle body and is adapted to detachably secure the handle body to the catheter. The medical device is sized to be deployed through the catheter. The wire is sized to pass through the lumen of the handle and the lumen of the catheter.

In yet another embodiment, a method of deploying a medical device is provided including positioning a distal end of the catheter at a deployment site, positioning the medical device in a lumen of the catheter, positioning a distal end of a wire within the lumen of the catheter, detachably coupling a handle to a proximal end of the catheter, and deploying the medical device within the body of a patient. The distal end of the wire is positioned proximally from the medical device. The handle includes a handle body, a first wheel, and a second wheel. The handle body includes an inner surface which defines a lumen extending from a distal end to a proximal end of the handle body. The first wheel is coupled to a first side of the handle body. The second wheel is coupled to a second side of the handle body. A portion of the second wheel is adapted to extend into the lumen of the handle body. The wire extends through the lumen of the handle body. The first wheel is movable from a release position to an engagement position. In the engagement position, the first wheel contacts the wire. Rotation of the first wheel or second wheel advances the wire distally through the distal end of the catheter to deploy the medical device.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIGS. 1-4 illustrate a series of cross-sectional views of a first example of method of deploying a medical device through a catheter utilizing a wire and a handle;

FIGS. 5-8 illustrate a series of cross-sectional views of a second example of method of deploying the medical device through the catheter utilizing the wire, a sleeve, and the handle;

FIG. 9 illustrates a side plan view of a third example of the wire with a first wheel and a second wheel;

FIG. 10 illustrates a cross-sectional view of an example of a coupling mechanism between the handle and a hub of the catheter;

FIG. 11 illustrates a cross-sectional view of a fourth example of the handle including the first wheel and the second wheel;

FIG. 12 illustrates a cross-sectional view of a fifth example of the handle including the first wheel and the second wheel; and

FIG. 13 illustrates a flow diagram of operations to deploy a medical device through a catheter.

The drawings described herein are for illustration purposes only and are not intended to limit the scope of the present disclosure in anyway.

### DETAILED DESCRIPTION

The following description is merely exemplary in nature and is not intended to limit the present disclosure, application, or uses.

In one example, a handle is provided for advancing a wire through a catheter, the handle including a handle body, a first wheel, a second wheel, and a coupling mechanism. The handle body has an outer surface and an inner surface. The inner surface defines a lumen extending along a longitudinal axis from a proximal end to a distal end of the handle body. The first wheel is coupled to a first side of the handle body. A portion of the first wheel is movable toward the longitudinal axis into a first lumen side of the lumen to an engagement position to contact a wire extending through the lumen. A second wheel is coupled to a second side of the handle body. A portion of the second wheel is adapted to extend into a second lumen side of the lumen which is different from the first lumen side. The coupling mechanism is at the distal end of the handle body and is adapted to detachably secure the handle body to the catheter.

One technical advantage of the systems and methods described below may be that precise control of the advancement of the wire minimizes the risk that the wire will kink during advancement. Another technical advantage may be that simultaneous manipulation of the catheter and wire from the handle minimizes the risk of off-target delivery.

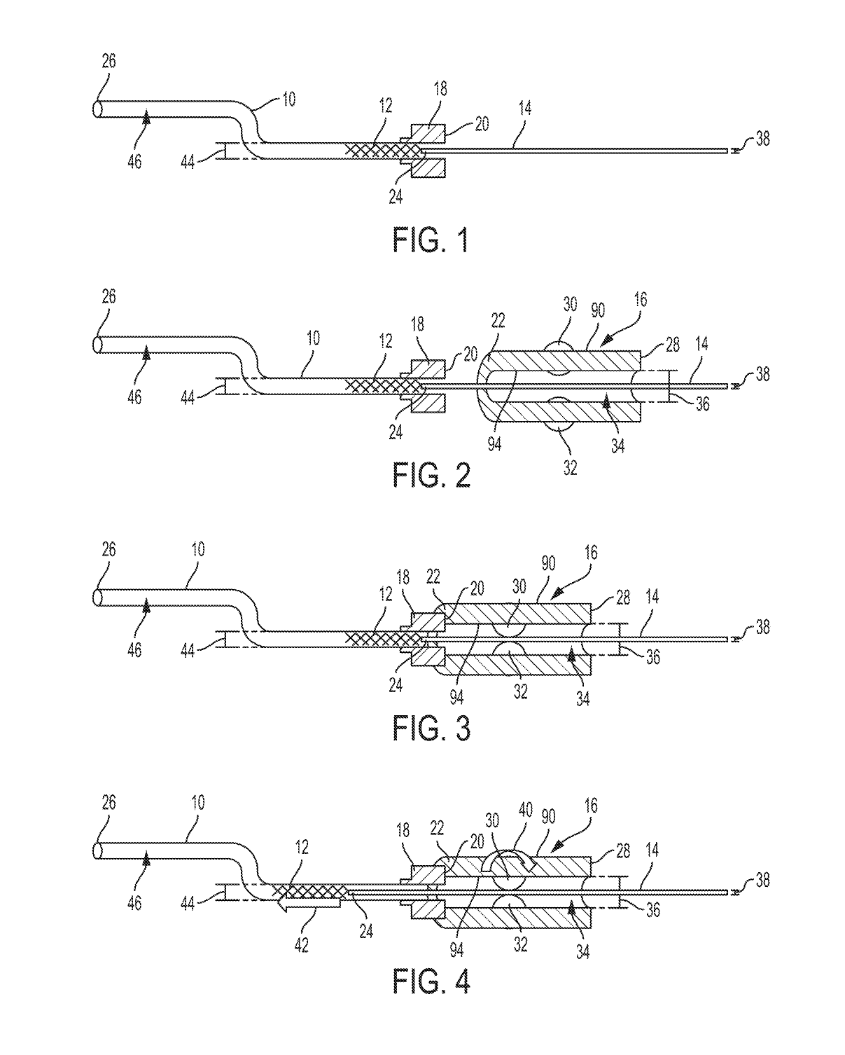

FIGS. 1-4 illustrate a series of cross-sectional views of a first example of a method of deploying a medical device 12 through a catheter 10 utilizing a wire 14 and a handle 16. The catheter 10 may be any object having a lumen 46 and which is configured to be placed in an endovascular passageway of a patient. Examples of the catheter 10 may include catheters, tubes, microcatheters, and sheaths. The catheter 10 may have a hub 18 located at a proximal end 20 of the catheter 10, the hub 18 being any device that facilitates connection between the catheter 10 and another component, such as a hemostasis valve. The medical device 12 may be any device which is configured to be deployed within an endoluminal passageway of a body of patient. Examples of the medical device 12 may include stents, embolism coils, and grafts. The medical device 12 may be expandable such that it has a compressed diameter to fit within the lumen 46 of the catheter and a larger, expanded diameter once it has been deployed in the endoluminal passageway.

The wire 14 may be any object configured to extend through the lumen 46 of the catheter 10 to push the medical device 12 through the catheter 10. Examples of the wire 14 may include a wire guide, a push rod, or a plunger. The medical device 12 may be detachably coupled to a distal end 24 of the wire 14. In some embodiments, the wire 14 and medical device 12 may be pre-loaded into the lumen 46 of the catheter 10 before the catheter 10 is positioned within the endoluminal passage. The wire 14 may have a diameter 38 which is less than a diameter 44 of the lumen 46 of the catheter 10. To ensure even distal advancement of the medical device 12 through the lumen 46, in some embodiments, the diameter 38 of the wire 14 may be as close in size as possible, the diameter 44 of the lumen 46, the diameter 38 of the wire 14 being for example between 50% and 99% of the diameter 44 of the lumen 46.

As shown in FIG. 1, the wire 14 may be positioned within the lumen 46 of the catheter 10 and located proximally from a position of the medical device 12. In some embodiments, the wire 14 and the medical device 12 may be pre-loaded into the lumen 46 of the catheter 10 before the catheter 10 is inserted into the body of the patient.

As shown in FIG. 2, a handle 16 may be advanced over the wire 14 and detachably coupled to the hub 18 to the catheter 10. The handle 16 may be any component which may facilitate movement of the wire 14 relative to the catheter 10. The handle 16 may include an inner surface 94, a first wheel 30, a second wheel 32, and a coupling mechanism 98 (FIG. 10) to connect a distal end 22 of the handle 16 to the hub 18 at the proximal end 20 of the catheter 10. The inner surface 94 extends between the proximal end 28 and the distal end 22 of the handle 16 and defines a lumen 34 extending therethrough.

The first and second wheels 30, 32 may be any components which, when positioned within a lumen 34 are configured to frictionally grip the wire 14 to advance or retract the wire 14. Examples of the first and second wheels 30, 32 may include cylindrical wheels, spherical wheels or polygonal shapes approximating a round shape.

The first wheel 30 may be coupled to a first side of the handle body 16 and may be movable between a release position and an engagement position. As shown in FIG. 2, in the release position, the first wheel 30 may extend outwardly from the outer surface 90 of the handle body 16 and may extend inwardly from the inner surface 94 of the handle body 16. In some embodiments, where the first wheel 30 extends inwardly from the inner surface 94 and into a first side of the lumen 34 in the release position, the first wheel 30 may be positioned to minimize contact with the wire 14 to allow uninhibited relative motion between the wire 14 and the handle body 16. In some embodiments, the first wheel 30 does not extend inwardly into the lumen 34 while in the release position.

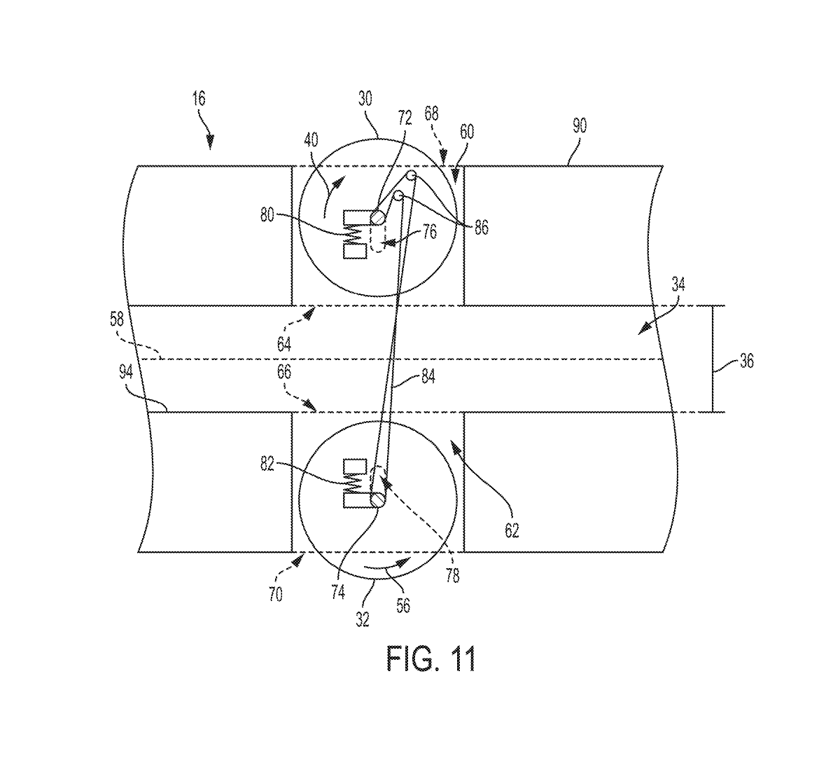

The second wheel 32 may be coupled to a second side of the handle body 16 and may be movable between a release position and an engagement position. As shown in FIG. 2, in the release position, the second wheel 32 may extend outwardly from the outer surface 90 of the handle body 16 and may extend inwardly from the inner surface 94 of the handle body 16. In some embodiments, where the second wheel 32 extends inwardly from the inner surface 94 and into a second side of the lumen in the release position, the second wheel 32 may be positioned to minimize contact with the wire 14 to allow uninhibited relative motion between the wire 14 and the handle body 16. In some embodiments, the second wheel 32 does not extend inwardly into the lumen 34 while in the release position. The second side of the handle body 16 may be different from the first side of the handle body 16. The first side may be longitudinally or angularly offset from the second side. In some embodiments, the first side and the second side of the handle body and the first side and the second side of the lumen 34 are each opposed to one another such that the first wheel 30 and second wheel 32 are positioned on opposing sides of a longitudinal axis (58 in FIG. 11) of the handle body 16.

As illustrated in FIG. 3, the distal end 22 of the handle body 16 may be detachably coupled to the proximal end 20 of the catheter 10. In some embodiments, the distal end 22 of the handle body 16 may be secured to the proximal end 20 of the catheter 10 by detachably coupling the distal end 22 of the handle body to the hub 18 which is coupled to the catheter 10. In some embodiments, the lumen 34 of the handle body 16 may have a diameter 36 which is similar or equal to the diameter 44 of the lumen 46 of the catheter 10 to provide a smooth transition between lumen 34 of the handle body 16 and the lumen 46 of the catheter 10 such that the wire 14 will not catch on an edge as it is being advanced into the proximal end 20 of the catheter 10.

Also illustrated in FIG. 3, the first wheel 30 and the second wheel 32 may be moved from the release position toward the longitudinal axis 58 into the engagement position. Where a wire 14 is present within the lumen 34 of the handle body 16, the first wheel 30 and the second wheel cooperatively contact the wire 14 in the engagement position.

As shown in FIG. 4, once the first wheel 30 and the second wheel 32 have gripped the wire 14, rotation 42 of one of the first wheel 30 or the second wheel 32 may cause longitudinal movement 42 of the wire 14 through the lumen 34 of the handle body 16 and through the lumen 46 of the catheter 10. If the medical device 12 is coupled to the wire 14, rotation 42 of one of the first wheel 30 or the second wheel 32 may cause longitudinal movement 42 of the medical device 12. The wire 14 may be advanced until the medical device 12 is pushed entirely through a distal end 26 of the catheter 10. In some embodiments, particularly wherein the medical device 12 is self-expanding, the deployment of the medical device 12 may be controlled by advancing the wire 14 in stages to push successive portions of the medical device 12 out of distal end 26 of the catheter 10. Finely controlling the advancement and retraction 42 of the wire 14 by rotation 40 of at least one of the first wheel 30 and the second wheel 32 may be critical in ensuring a complete deployment of the medical device 12 in a particular area of the endovascular passage. After the medical device 12 has been deployed, the wire 14 may be fully retracted from the lumen 46 of the catheter 10 and the handle 16 may be detached from the catheter 10.

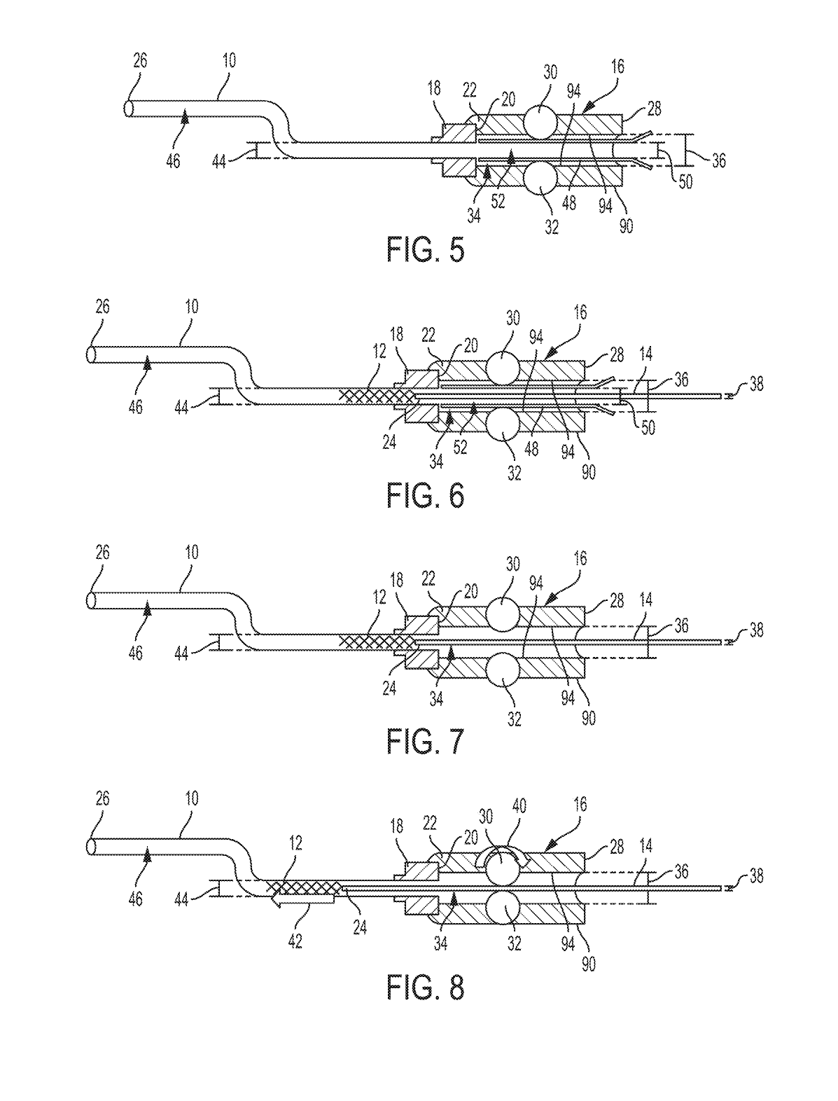

FIGS. 5-8 illustrate a series of cross-sectional views of a second example of a method of deploying a medical device 12 through a catheter 10 utilizing a wire 14 and a handle 16. As show in FIG. 5, the handle body 16 may be coupled to the catheter 10 or the hub 18 before the wire 14 and the medical device 12 are inserted into the lumen 46 of the catheter 10. In some embodiments, a sleeve 48 may be positioned within the lumen 34 of the handle body 16 such that the wire 14 and the medical device 12 may be advanced through the lumen 34 of the handle body 16 without contacting either of the first wheel 30 or the second wheel 32. The sleeve 48 may be any object which is sized to be positioned in the lumen 34 and which prevents movement of the first wheel 30 and second wheel 32 toward the engagement position. Examples of the sleeve 48 may include an open-ended cylinder, or any other shape through which the wire 14 and the medical device 12 may pass. As shown in FIG. 5, the sleeve 48 may have a flared proximal end to allow for easier insertion and compression of the medical device 12 and the wire 14 into the lumen 34 of the handle body 16.

The sleeve 48 may have an inner diameter 50 which is similar or equal to the diameter 44 of the lumen 46 of the catheter 10 to provide a smooth transition between the lumen 34 of the handle body 16 and the lumen 46 of the catheter 10 such that the wire 14 will not catch on an edge as it is being advanced into the proximal end 20 of the catheter 10. In such an embodiment, the diameter 36 of the lumen 34 may be slightly larger than the diameter 44 of the lumen 46 of the catheter 10 to accommodate the sleeve 48.

As illustrated in FIG. 6, the medical device 12 and wire 14 may be inserted into the catheter 10 through the sleeve 48 positioned in the handle body 16. The medical device 12 and wire may be advanced distally up to a position where fine control may be needed to further advance the medical device 12.

Once the wire 14 and medical device 12 have been inserted into the catheter 10 through the lumen 34 of the handle body 16, the sleeve 48 may be removed, as shown in FIG. 7. The sleeve 48 may be removed by retracting the sleeve 48 over the wire 14. In other embodiments, the sleeve 48 may include a perforation or a pull string to allow the sleeve 48 to be removed by tearing a seam in the sleeve 48. Once the sleeve 48 has been removed from the handle body 16, the first wheel 30 and the second wheel 32 may be moved to the engagement position to control the movement 42 of the medical device 12 and the wire 14.

In some embodiments, the first wheel 30 and the second wheel 32 are movable from the release position to the engagement position by an inward force being applied to the wheel from the outer surface 90 of the handle body. For example, an operator may use a finger to press one of the first wheel 30 and the second wheel 32 inward toward the longitudinal axis 58 to the engagement position. In embodiments where the sleeve 48 is not present in the lumen 34, the first wheel 30 and the second wheel 32 may be biased in the release position to reduce undesirable interference between the first and second wheels 30, 32 and the wire 14. Where the handle body 16 includes the sleeve 48, the first wheel 30 and second wheel 32 may be biased toward the engagement position such that, when the sleeve 48 is removed, no additional external force is needed to move the first and second wheels 30, 32 to the engagement positions. In such an embodiment, the sleeve 48 may be reintroduced into the lumen 34 of the handle body 16 to move the first and second wheels 30, 32 back toward the release position so that the wire 14 can be retracted.

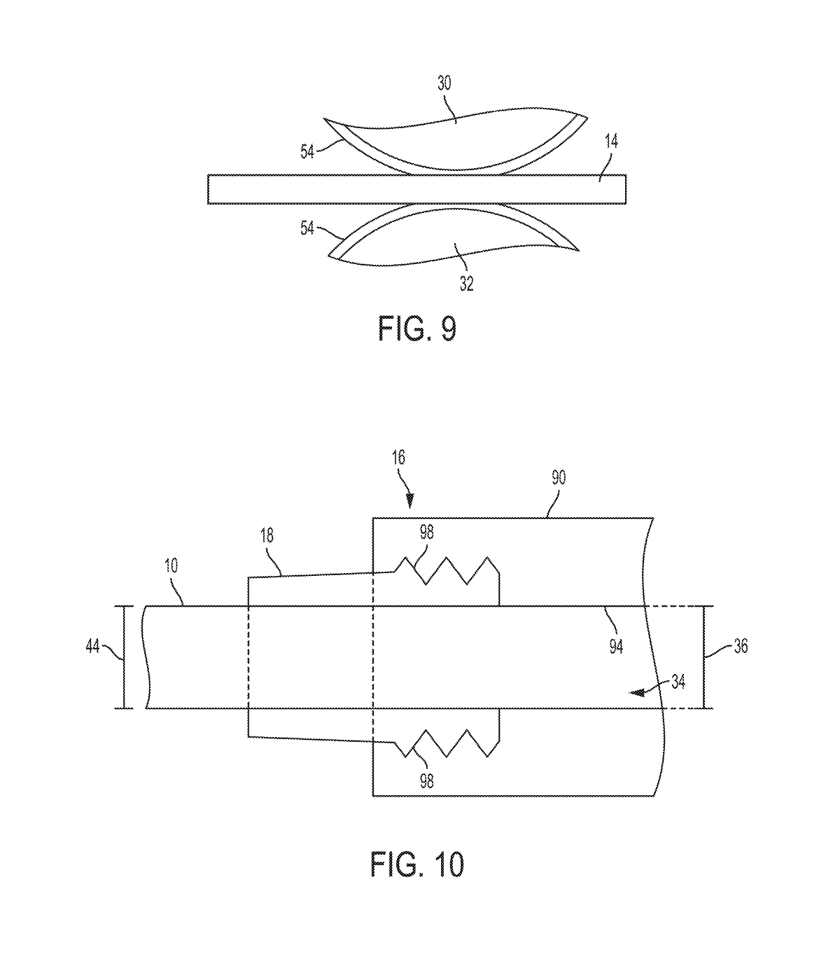

As illustrated in FIG. 9, the outer surface 54 of the first wheel 30 and the second wheel 32 may include a high friction, low durometer material to grip the wire 14 while in the engagement position. The entirety of the first and second wheels 30, 32 may be made of such a material or only a portion of the first and second wheels 30, 32 may be made of such a material. The outer surface 54 may be made of any material having a low durometer and a high frictional coefficient. Examples of materials for the outer surface 54 include silicone, polyurethane, and other suitable polymers. The durometer of the outer surface 54 may be between 10 A and 80 A (on the Shore A Hardness Scale). The frictional coefficient of the outer surface may be between 0.5 and 1.2.

As illustrated in FIG. 10, the coupling mechanism 98 which allows the detachable coupling of the catheter 10 to the handle body 16 may include a threaded surface on the outer surface of the proximal end 20 of the catheter 10 or the hub 18 and a matching threaded surface on the inner surface 94 of the handle body 16 which is adapted to mate with the threaded surface of the catheter 10 or hub 18. Alternatively, in some embodiments, the catheter 10 or hub 18 may include an interior threaded surface which couples with an exterior threaded surface on the handle body 16. Other coupling mechanisms 98 may be used such as a groove and slot configuration, or a male and female luer connection.

FIG. 11 illustrates a cross-sectional view of an example of the interior of the handle body 16. The rotational motion 40 of the first wheel 30 may be linked to the rotational movement 56 of the second wheel 32. The first wheel 30 may rotate about a first axle 72 and the second wheel 32 may rotate about a second axle 74. A linking mechanism 84 may extend between the first axle 72 and the second axle 74 such that when the first wheel 30 rotates in a clockwise direction to advance the wire 14, the second wheel 32, by the linking mechanism 84, rotates in an opposite direction, a counter clockwise direction, to cooperatively advance the wire 14. Examples of the linking mechanism may include a geared system or a pulley 86 and cable 84 system as shown in FIG. 11.

The movement of the first wheel 30 and the second wheel 32 toward and away from the longitudinal axis 58, between the release position and the engagement position, may also be linked. The first wheel 30 may be coupled to a first side of the handle body 16 in a first wheel housing 60 such that a portion of the first wheel 30 extends through a first opening 68 in the outer surface 90 of the handle body 16 in both the release position and the engagement position. The first axle 72 of the first wheel 30 may be disposed within a first slot 76 in the first wheel housing 60 to define the movement of the first wheel 30 such that when the first axle 72 is in a first end of the first slot 76, the first wheel 30 is in the release position (as shown in FIG. 11), and when the first axle 72 is in a second end of the first slot 76, the first wheel 30 is in the engagement position. The first wheel 30 may be moveable such that in the release position, the first wheel 30 does not occlude any portion of the lumen 34, and when the first wheel 30 is in the engagement position, a portion of the first wheel 30 extends through a first opening 64 in the inner surface 94 of the handle body 16 and into the lumen 34.

The second wheel 32 may be coupled to a second side of the handle body 16 in a second wheel housing 62 such that a portion of the second wheel 32 extends through a second opening 70 in the outer surface 90 of the handle body 16 in both the release position and the engagement position. The second axle 74 of the second wheel 32 may be disposed within a second slot 78 in the second wheel housing 62 to define the movement of the second wheel 32 such that when the second axle 74 is in a first end of the second slot 78, the second wheel 32 is in the release position (as shown in FIG. 11), and when the second axle 74 is in a second end of the second slot 78, the second wheel 32 is in the engagement position. The second wheel 32 may be moveable such that in the release position, the second wheel 32 does not occlude any portion of the lumen 34, and when the second wheel 32 is in the engagement position, a portion of the second wheel 32 extends through a second opening 66 in the inner surface 94 of the handle body 16 and into the lumen 34.

As illustrated in FIG. 11, movement of the first wheel 30 and the second wheel 32 from the release position to the engagement position may be linked by the linking mechanism 84 such that when one of the first wheel 30 and the second wheel 32 is moved toward the engagement position, the other of the first wheel 30 and the second wheel 32 also moves toward the engagement position. Similarly, when one of the first wheel 30 and the second wheel 32 is moved toward the release position, the other of the first wheel 30 and the second wheel 32 also may move toward the release position. In the example shown in FIG. 11, the linking mechanism is an elastic band 84 supplemented by a first biasing member 80, a second biasing member 82, and two rotational pulleys 86. The elastic band 84 may extend to the rotational pulleys 86 located outward from the lumen 34 relative to the first axle 72 and the second axle 74. The first biasing member 80 exerts a force to bias the first wheel 30 toward the release position. The second biasing member 82 exerts a force to bias the second wheel 32 toward the release position. When an external force is applied to one of the first wheel 30 or second wheel 32 from the outer surface 90, the respective first wheel 30 or second wheel 32 may move toward the engagement position, increasing tension in the elastic band 84. The increased tension in the elastic band 84 may exert a force causing the other of the first wheel 30 or second wheel 32 to also move toward the engagement position. In the embodiment shown in FIG. 11, the linking mechanism 84 links both the rotation of the first wheel 30 and second wheel 32 and the movement of the first wheel 30 and second wheel 32 toward and away from the engagement position. However, in other embodiments, multiple linking mechanisms 84 may be present to separately control these two linked movements between the first wheel 30 and the second wheel 32.

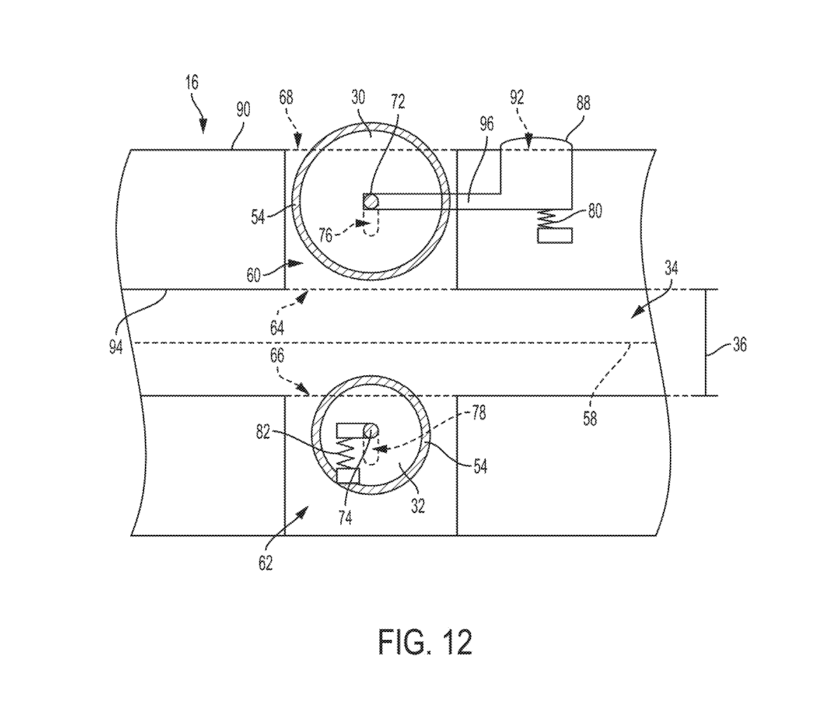

FIG. 12 illustrates a cross-sectional view of another example of the handle body 16. As shown in FIG. 12, the first axle 72 is coupled to a button 88 through a button arm 96. The button 88 is movable through an opening 92 in the outer surface 90 of the handle body 16 between the release position (shown in FIG. 11) and the engagement position. The button 88 may be depressed to move toward the engagement position, causing the first wheel 30 to also move toward the engagement position. In some embodiments, the button 88 may be locked in the engagement position, allowing advancement of the wire 14 by rotation of the first wheel 30 by applying a rotational force to the portion of the first wheel 30 which extends beyond the outer surface 90 of the handle body 16. The button 88 may be biased in the release position by the first biasing member 80.

In some embodiments, the second wheel 32 may not extend outward beyond the outer surface 90 of the handle body 16. In such an embodiment, the second wheel 32 may have a smaller diameter than the first wheel 30. Additionally, in such an embodiment, the second wheel 32 may be biased toward the engagement position by the second biasing member 82. In such an embodiment, when the first wheel 30 is in the release position, the second wheel 32 may spin freely against the wire 14 as it is advanced through the lumen 34 of the handle body 16, causing minimal impedance to the movement of the wire 14. When the first wheel 30 is moved to the engagement position, the wire 14 may be gripped by the force applied to the button 88 through the first wheel 30 on the first side of the lumen 34 and by the force applied by the second biasing member 82 through the second wheel 32 on the second side of the lumen 34. In such a configuration, clockwise rotation of the first wheel 30 may cause advancement of the wire 14 and counterclockwise rotation of the second wheel 32.

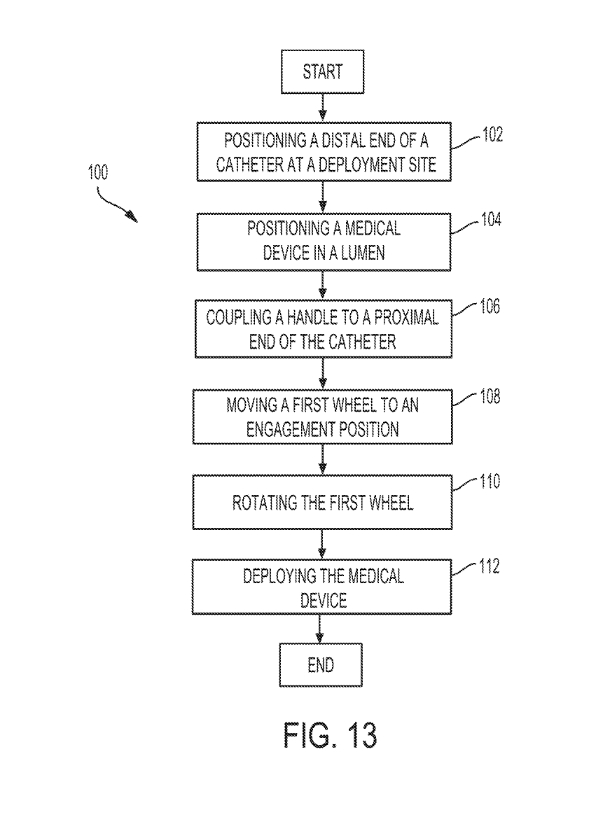

FIG. 13 illustrates a flow diagram of operations to deploy the medical device 12 through the catheter 10 (100). The operations (100) may include fewer, additional, or different operations as illustrated in FIG. 13. Alternatively or in addition, the operations (100) may be performed in a different order than illustrated.

Initially, the method of operations (100) includes positioning the distal end 26 of the catheter 10 at a deployment site within a body of a patient (102). The method of operations (100) also includes positioning the medical device 12 in the lumen 46 of the catheter 10 (104). The method further includes positioning the distal end 24 of the wire 14 within the lumen 46 of the catheter 10 proximally from the medical device. The method of operations (100) further includes detachably coupling the handle body 16 to the proximal end 20 of the catheter 10 (106). These steps may be performed concurrently or separately. For example, in some embodiments, the medical device 12 and the wire 14 may be positioned within the lumen 46 catheter 10 (104) before the catheter 10 is positioned at the delivery site (102). In other embodiments, the handle body 16 is detachably coupled to the proximal end 20 of the catheter 10 (106) before the wire 14 and medical device 12 are advanced into the lumen 46 of the catheter 10.

The method of operations (100) further includes moving the first wheel 30 from a release position to an engagement position such that an outer surface 54 of the first wheel 30 contacts the wire 14 (108). In some embodiments, the second wheel 32 also contacts the wire 14. The method of operations (100) further includes rotating one of the first wheel 30 or the second wheel 32 to advance the wire 14 distally toward the distal end 26 of the catheter 10 (110). The method of operations (100) further includes deploying the medical device 12 within the body of the patient (112) by advancing the wire 14 distally against the medical device 12 until the medical device 12 has advanced through the distal end 26 of the catheter 10.

In addition to the advantages that have been described, it is also possible that there are still other advantages that are not currently recognized but which may become apparent at a later time. While various embodiments have been described, it will be apparent to those of ordinary skill in the art that many more embodiments and implementations are possible. Accordingly, the embodiments described herein are examples, not the only possible embodiments and implementations.

What is claimed is:

1. A handle for advancing a wire through a catheter, the handle comprising:
a handle body having an outer surface and an inner surface, the inner surface defining a lumen extending along a longitudinal axis extending from a proximal end to a distal end of the handle body;
a first wheel coupled to a first side of the handle body, a portion of the first wheel being movable toward the longitudinal axis to an engagement position on a first lumen side within the lumen to contact the wire;
a second wheel coupled to a second side of the handle body, wherein a portion of the second wheel is adapted to extend into the lumen on a second lumen side different from the first lumen side; and
a coupling mechanism at the distal end of the handle body adapted to detachably secure the handle body to the catheter.

2. The handle of claim 1, wherein a second portion of the first wheel in the engagement position extends outwardly from the outer surface of the handle body.

3. The handle of claim 1, wherein the first wheel and the second wheel are positioned radially along opposite sides relative to the longitudinal axis.

4. The handle of claim 1, wherein the first wheel and the second wheel are rotatably linked such that rotation in a first direction of one of the first wheel and the second wheel causes the rotation in a second opposite direction of the other of the first wheel and the second wheel.

5. The handle of claim 1, wherein the first wheel is movable between a release position and the engagement position, wherein the second wheel is movable between a release position and an engagement position, and wherein the second wheel is adapted to extend into the lumen only while in the engagement position to contact the wire.

6. The handle of claim 5, wherein movement of the first wheel and the second wheel are cooperatively linked such that movement of one of the first wheel and the second wheel from the release position to the engagement position will cause the other of the first wheel and the second wheel to move from the release position to the engagement position.

7. The handle of claim 1, wherein the first wheel comprises a high friction, low durometer outer surface.

8. The handle of claim 7, wherein the outer surface of the first wheel is silicone.

9. The handle of claim 1, wherein the first wheel is biased in a release position away from the longitudinal axis relative to the engagement position.

10. The handle of claim 1, wherein the first wheel is biased in the engagement position.

11. The handle of claim 10, further comprising a sleeve removably positioned within the lumen to prevent movement of the first wheel to the engagement position.

12. A system for deploying a medical device through a catheter, comprising:
a catheter having a lumen;
a handle comprising a handle body having an inner surface defining a lumen about a longitudinal axis extending from a proximal end to a distal end of the handle body, a first wheel coupled to a first side of the handle body, the first wheel being movable toward the longitudinal axis into a first lumen side of the lumen to an engagement position, a second wheel coupled to a second side of the handle body, wherein a portion of the second wheel is adapted to extend into a second lumen side of the lumen, and a coupling mechanism at the distal end of the handle body adapted to detachably secure the handle body to the catheter;
a medical device sized to be deployed through the catheter; and
a wire sized to pass through the lumen of the handle and the lumen of the catheter.

13. The system of claim 12, wherein the coupling mechanism of the handle comprises a threaded surface at the distal end of the handle body, the threaded surface adapted to mate with a threaded surface at a proximal end of the catheter.

14. The system of claim 12, wherein a diameter of the lumen of the catheter is sized to match a diameter of a lumen of the handle.

15. A method of deploying a medical device through a catheter comprising:
positioning a distal end of the catheter at a deployment site within a body of a patient, the catheter comprising a lumen;
positioning the medical device in the lumen of the catheter;
positioning a distal end of a wire within the lumen of the catheter proximally from the medical device;
detachably coupling a handle to a proximal end of the catheter, the handle comprising a handle body comprising an inner surface defining a lumen extending from a proximal end to a distal end of the handle body, a first wheel coupled to a first side of the handle body, a second wheel coupled to a second side of the handle body, wherein a portion of the second wheel is adapted to extend into the lumen of the handle body, wherein the wire extends through the lumen of the handle body;
moving the first wheel from a release position to an engagement position such that an outer surface of the first wheel contacts the wire;
rotating one of the first wheel or the second wheel to advance the wire distally toward the distal end of the catheter; and
deploying the medical device within the body of the patient.

16. The method of claim 15, wherein the handle is coupled to the proximal end of the catheter before the wire is positioned within the catheter.

17. The method of claim 16, wherein the handle further comprises a sleeve removably positioned within the lumen of the handle body to prevent movement of the first wheel to the engagement position.

18. The method of claim 17, further comprising removing the sleeve from the lumen of the handle body before moving the first wheel to the engagement position.

19. The method of claim 18, further comprising inserting the sleeve into the lumen of the handle body after deploying the medical device.

20. The method of claim 19, further comprising, removing the wire from the lumen of the handle body after inserting the sleeve into the lumen.
